SEQUENCE LISTING (7 sequences)

SEQ ID NO: 1 - protein, 146 aa, Homo sapiens
MHPLLNPLLLALGLMALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 2 - protein, 81 aa, Homo sapiens
MVWSINLTAGMYCAALESLINVSGCSAIEKTQRMLSGFCPHKVSAGQFSSLHVRDTKIEVAQFVKDLLLHLKKLFREGQFN

SEQ ID NO: 3 - protein, 131 aa, Mus musculus
MALWVTAVLALACLGGLAAPGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPF

SEQ ID NO: 4 - protein, 153 aa, Homo sapiens [sequence omitted]

SEQ ID NO: 5 - protein, 137 aa, Homo sapiens
MGLTSQLLPPLFFLLACAGNFVHGHKCDITLQEIIKTLNSLTEQKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 6 - protein, 136 aa, Homo sapiens
MGLTSQLLPPLFFLLACAGNFVHGHKCDITLQEIIKTLNSLTEQKTLCTELTVTDIFAASKQHACLPTSRHTGTICRPPSARVPSTSTRLQSPERTQLRRKPSAGLRLCSGSSTATMRRTLAAWVRLHSSSTGTSS

SEQ ID NO: 7 - protein, 139 aa, Mus musculus
MGLNPQLVVILLFFLECTRSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESSTSLKDFLESLKSIMQMDYS

(19) United States
(12) Patent Application Publication
(10) Pub. No.: US 2022/0378877 A1
(43) Pub. Date: Dec. 1, 2022

(54) TREATMENT OF CLOSTRIDIUM DIFFICILE INFECTIONS

(71) Applicant: University of Virginia Patent Foundation, Charlottesville, VA (US)

(72) Inventors: William A. Petri, JR., Charlottesville, VA (US); Alexandra N. Donlan, Charlottesville, VA (US)

(21) Appl. No.: 17/776,524

(22) PCT Filed: Nov. 13, 2020
(86) PCT No.: PCT/US20/60561

§ 371 Date: May 12, 2022

Related U.S. Application Data
(60) Provisional application No. 62/934,871, filed on Nov. 13, 2019.

(51) Int. Cl. A61K 38/20; A61P 31/04; C07K 16/28; A61K 45/06; A61K 38/14; A61K 31/7048; A61K 31/4164; A61K 31/426; A61K 31/437

(52) U.S. Cl. CPC A61K 38/2086; A61P 31/04; C07K 16/2866; A61K 45/06; A61K 38/2026; A61K 38/20; A61K 38/14; A61K 31/7048; A61K 31/4164; A61K 31/426; A61K 31/437; C07K 2317/76

(57) ABSTRACT

Methods and compositions for treating or preventing C. difficile infections, particularly recurring C. difficile infections are described. The compositions for use in treating C. difficile include at least one agent that enhances a biological activity of interleukin-13 (IL-13), such as a recombinant IL-13 peptide or an agent that neutralizes the interleukin-13 (IL-13) decoy receptor, interleukin-13 receptor subunit alpha-2 (IL-13Ra2). Additionally or alternatively, the compositions can include a interleukin-4 (IL-4) peptide. The methods can result in more rapid recovery from CDI and decreased weight loss, e.g., than treatment without the neutralizing agent.

### CROSS REFERENCE TO RELATED APPLICATION

The presently disclosed subject matter claims the benefit of U.S. Provisional Patent Application Ser. No. 62/934,871, filed Nov. 13, 2019, the disclosure of which incorporated herein by reference in its entirety.

### GOVERNMENT SUPPORT

This invention was made with government support under Grant Number A1124214 awarded by the National Institutes of Health. The government has certain rights in the invention.

### TECHNICAL FIELD

The presently disclosed subject matter relates generally to methods for treating or preventing Clostridium difficile infections, including recurring Clostridium difficile infections.

### BACKGROUND

Clostridium difficile is a gram-positive, spore-forming obligate anaerobe. It is the leading cause of hospital acquired gastrointestinal infection in the U.S. The observed rate of C. difficile infection (CDI) has risen from about 5 per 1,000 hospital discharges in 2001 to about 13 per 1,000 discharges in 2011. Risk factors for CDI include age and antibiotic usage.

In particular, antibiotics can disrupt the normal microbiota and allow for colonization by C. difficile. Large amounts of epithelial damage are mediated by the bacterial toxins, which include Toxins A and B, and the additional toxin CDT, present in the epidemic strain of C. difficile. Being an acute infection, the innate immune response is a factor behind host protection, involving neutrophils and proinflammatory factors such as IL-1β and IL-23, which can be damaging, in excess.

Accordingly, there is an ongoing need for additional methods of treating or preventing C. difficile infections.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides a method of treating or preventing a Clostridium difficile (C. difficile) infection, or a symptom or pathology associated therewith, in a subject in need thereof, the method comprising administering to the subject a composition that comprises a therapeutically effective amount of at least one agent that enhances a biological activity of interleukin-13 (IL-13) in the subject.

In some embodiments, the at least one agent that enhances a biological activity of IL-13 comprises an IL-13 peptide or a fragment or homolog thereof. In some embodiments, the IL-13 peptide is a peptide having an amino acid sequence that is at least 95% identical to SEQ ID NO: 1, SEQ ID NO: 2, or SEQ ID NO: 3.

In some embodiments, the at least one agent that enhances an activity of IL-13 comprises an agent that blocks interleukin-13 receptor subunit alpha-2 (IL-13Ra2). In some embodiments, the agent that blocks IL-13Ra2 is an antibody that binds to IL-13Ra2, optionally wherein the antibody that binds to IL-13Ra2 is a monoclonal antibody.

In some embodiments, the administering results in a more rapid recovery from a C. difficile infection as compared to the recovery expected in the absence of administering the composition to the subject. In some embodiments, the administering prevents a recurrence of a C. difficile infection.

In some embodiments, the subject is a mammal. In some embodiments, the subject is a human. In some embodiments, the subject has a C. difficile infection. In some embodiments, the subject has one or more increased risk factors for a C. difficile infection, optionally wherein the one or more increased risk factors are selected from the group comprising hospitalization, antibiotic use, and increased age.

In some embodiments, administering the composition inhibits weight loss. In some embodiments, administering the composition inhibits diarrhea. In some embodiments, administering the composition inhibits colonic inflammation.

In some embodiments, the method further comprises administering at least one additional therapeutic agent to the subject, optionally wherein the composition comprises the at least one additional therapeutic agent. In some embodiments, said at least one additional therapeutic agent is selected from the group comprising an anesthetic, an analgesic, an antimicrobial agent, a steroid, a growth factor, a cytokine other than IL-13, and an anti-inflammatory agent.

In some embodiments, said at least one additional therapeutic agent comprises a cytokine other than IL-13, optionally an interleukin other than IL-13, further optionally wherein said at least one additional therapeutic agent comprises interleukin-4 (IL-4), interleukin-33 (IL-33), or interleukin-25 (IL-25). In some embodiments, said at least one additional therapeutic agent comprises an antimicrobial agent, optionally wherein said antimicrobial agent is selected from the group comprising an antibacterial agent, an antifungal agent, and an antiviral agent. In some embodiments, said antimicrobial agent comprises at least one antibiotic selected from the group comprising vancomycin, fidaxomicin, metronidazole, nitazoxanide, and rifaximin.

In some embodiments, the presently disclosed subject matter provides a composition for use in treating or preventing a Clostridium difficile (C. difficile) infection, or a symptom or pathology associated therewith, wherein the composition comprises a therapeutically effective amount of at least one agent that enhances a biological activity of interleukin-13 (IL-13). In some embodiments, the agent that enhances a biological activity of IL-13 comprises an IL-13 peptide or a fragment or homolog thereof, optionally wherein the IL-13 peptide comprises an amino acid sequence that is at least 95% identical to SEQ ID NO: 1, SEQ ID NO: 2, or SEQ ID NO: 3. In some embodiments, the agent that enhances a biological activity of IL-13 comprises an agent that blocks interleukin-13 receptor subunit alpha-2 (IL-13Ra2), optionally wherein said agent is an anti-IL-13Ra2 antibody. In some embodiments, the composition further comprises at least one additional therapeutic agent, optionally wherein said at least one additional therapeutic agent is selected from the group comprising an anesthetic, an analgesic, an antimicrobial agent, a steroid, a growth factor, a cytokine other than IL-13, and an anti-inflammatory agent.

In some embodiments, the presently disclosed subject matter provides a method of treating or preventing a Clostridium difficile (C. difficile) infection, or a symptom or pathology associated therewith, in a subject in need thereof, the method comprising administering to the subject a composition that comprises a therapeutically effective amount of an interleukin-4 (IL-4) peptide, or a fragment or a homolog thereof, optionally wherein the IL-4 peptide comprises an amino acid sequence that is at least 95% identical to SEQ ID NO: 4, SEQ ID NO: 5, SEQ ID NO: 6, or SEQ ID NO: 7. In some embodiments, the administering results in a more rapid recovery from a C. difficile infection as compared to the recovery expected in the absence of administering the composition to the subject. In some embodiments, the administering prevents a recurrence of a C. difficile infection.

In some embodiments, the subject is a mammal. In some embodiments, the subject is a human. In some embodiments, the subject has a C. difficile infection. In some embodiments, the subject has one or more increased risk factors for a C. difficile infection, optionally wherein the one or more increased risk factors are selected from the group comprising hospitalization, antibiotic use, and increased age.

In some embodiments, administering the composition inhibits weight loss. In some embodiments, administering the composition inhibits diarrhea. In some embodiments, administering the composition inhibits colonic inflammation.

In some embodiments, the method further comprises administering at least one additional therapeutic agent to the subject, optionally wherein the composition comprises the at least one additional therapeutic agent. In some embodiments, said at least one additional therapeutic agent is selected from the group comprising an anesthetic, an analgesic, an antimicrobial agent, a steroid, a growth factor, a cytokine other than IL-4, and an anti-inflammatory agent. In some embodiments, said at least one additional therapeutic agent comprises a cytokine other than IL-4, optionally an interleukin other than IL-4, further optionally wherein said at least one additional therapeutic agent comprises interleukin-13 (IL-13), interleukin-33 (IL-33), or interleukin-25 (IL-25). In some embodiments, said at least one additional therapeutic agent comprises an antimicrobial agent, optionally wherein said antimicrobial agent is selected from the group comprising an antibacterial agent, an antifungal agent, and an antiviral agent. In some embodiments, said antimicrobial agent comprises at least one antibiotic selected from the group comprising vancomycin, fidaxomicin, metronidazole, nitazoxanide, and rifaximin.

In some embodiments, the presently disclosed subject matter provides a composition for use in treating or preventing a Clostridium difficile (C. difficile) infection, or a symptom or pathology associated therewith, wherein the composition comprises a therapeutically effective amount of an interleukin-4 (IL-4) peptide, or a fragment or a homolog thereof, optionally wherein the IL-4 peptide comprises an amino acid sequence that is at least 95% identical to SEQ ID NO: 4, SEQ ID NO: 5, SEQ ID NO: 6, or SEQ ID NO: 7. In some embodiments, the composition further comprises at least one additional therapeutic agent, optionally wherein said at least one additional therapeutic agent is selected from the group comprising an anesthetic, an analgesic, an antimicrobial agent, a steroid, a growth factor, a cytokine other than IL-4, and an anti-inflammatory agent.

Accordingly, it is an object of the presently disclosed subject matter to provide compositions and methods for treating or preventing C. difficile infections.

This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and Example. Additionally, various aspects and embodiments of the presently disclosed subject matter are described in further detail below.

### BRIEF DESCRIPTION OF THE FIGURES

FIG. 1 is a schematic diagram of C. difficile susceptibility and infection.

FIG. 2 is a schematic diagram showing the R20291 infection model.

FIG. 3A is a graph showing weight loss (as a percentage based on starting weight) of mice infected with 10⁴ R20291 spores and administered either neutralizing IL-13 or IgG control on days 0 and 2 post infection (DPI). N=10 mice per group, *<0.05, ***<0.0005.

FIG. 3B is a graph showing clinical scores of mice infected with 10⁴ R20291 spores and administered either neutralizing IL-13 or IgG control on days 0 and 2 post infection (DPI). N=10 mice per group, *<0.05, **<0.0005.

FIG. 4A is a graph showing percentages of neutrophils, Ly6C-low monocytes and Ly6C-high monocytes as a percent of total CD11B+ myeloid cells in mice were infected with 500 spores of R20291 and administered either anti-IL-13 or IgG control i.p. injections on days 0 and 2 post infection (DPI). N=10 mice per group, *<0.05, **<0.01, ***<0.001.

FIG. 4B is a graph showing total numbers of neutrophils, Ly6C-low monocytes and Ly6C-high monocytes in mice were infected with 500 spores of R20291 and administered either anti-IL-13 or IgG control i.p. injections on days 0 and 2 post infection (DPI). N=10, mice per group *<0.05, **<0.01, ***<0.001

FIG. 4C is a graph showing total numbers of CD45 and CD11B+ cells in mice infected with 500 spores of R20291 and administered either anti-IL-13 or IgG control i.p. injections on days 0 and 2 post infection. N=10 mice per group, ** p>0.01.

FIG. 5 is a schematic diagram showing a protocol for studying the effects of recombinant interleukin-13 (rIL-13) on the R20291 infection model.

FIG. 6A is a graph showing weight loss (as a percentage based on starting weight) of mice infected with 10⁴ R20291 spores and administered IL-13, IL-4, or phosphate buffered saline (PBS) control on days 0 and 1 post infection (DPI). N=10 mice per group. *<0.05, **<0.005.

FIG. 6B is a graph showing clinical scores of mice infected with 10⁴ R20291 spores and administered either interleukin-13 (IL-13), interleukin-4 (IL-4) or phosphate buffered saline (PBS) control on days 0 and 1 post infection (DPI). N=10 mice per group. *<0.05, **<0.005.

FIG. 7 is a schematic drawing showing a model of Type 2 immunity in the colon and the deleterious role of IL-13Ra2 therein.

FIG. 8A is a graph showing cecal IL-13Ra2 protein expression (in picograms per milliliter per milligram (pg/ml/mg)) in mice treated with antibiotics only (Antibiotics) or treated with antibiotics and infected with 500 spores of R20291 were measured via ELISA. IL-13Ra2 protein expression levels in the infected mice were measured on day 2, day 3 and day 5 post infection. For comparison, protein expression is also shown for mice that were not infected and that did not receive any antibiotics.

FIG. 8B is a graph showing the relationship between mouse weight (shown as a percentage of starting weight) and IL-13Ra2 protein expression (in (in picograms per milliliter per milligram (pg/ml/mg)).

FIG. 9A is a graph showing the total number of CD206+ M2-like macrophages in mice treated with antibiotics and infected with R20291 spores. These macrophages increase during recovery from infection (day 2 and day 5 post infection). N=10 mice per group, * <0.05.

FIG. 9B is a graph showing CH206+ M2-like macrophages as a percent of total CD11B+ myeloid cells in mice treated with antibiotics and infected with R20291 spores. The percent of these macrophages first decreases following infection (day 2) and then increases as recovery progresses (day 5). N=10 mice per group, * <0.05.

FIG. 10A is a pair of graphs showing how treatment with neutralizing interleukin-13 (anti-IL-13) reduces the number of CD206+ macrophages by 3 days post infection with R20291 spores compared to treatment with control antibody (IgG). The graph on the left shows the number of CD206+ macrophages as a percent of CD64+ macrophages, while the graph on the right shows the total cell count. N=10 mice per group, ** <0.005, *** <0.0005.

FIG. 10B is a pair of graphs showing the fluorescence-activated cell sorting (FACS) analysis for the CD206+ macrophages used to provide the data in FIG. 10A. The graph on the left is from the control group, while the graph on the right is from the group treated with neutralizing interleukin-13.

FIG. 11 is a graph showing that CCR2+ cell depletion results in more severe disease in mice. Treatment with interleukin-13 (IL-13) can promote CD 206+ macrophage polarization from tissue-recruited CCR2+ monocytes. When these monocytes are depleted starting on day 2 post-infection, mice have worse disease.

FIG. 12 is a graph showing the correlation between interleukin-13 (IL-13) and the anti-inflammatory cytokine interleukin-10 (IL-10) in mice treated with antibiotics (Abx) and infected with 500 R20291 spores. Cecal IL-13 and IL-10 protein expression (in picograms per milliliter per milligram (pg/ml/mg)) in mice treated with antibiotics only (Abx) or treated with antibiotics and infected with 500 spores of R20291 were measured via ELISA. IL-13 and IL-10 protein expression levels in the infected mice were measured on day 2, day 4 and day 5 post infection. For comparison, protein expression is also shown for mice that were not infected and that did not receive any antibiotics (control).

### DETAILED DESCRIPTION

The presently disclosed subject matter is based in part on the finding that a type 2 immune response is protective, likely by mitigating damaging inflammation and tissue damage. Both interleukin-25 (IL-25) and interleukin-33 (IL-33) have been shown to be protective, through eosinophils and Type 2 Innate Lymphoid Cells (ILC2), respectively. Neutralization of interleukin-4 (IL-4) and, as shown herein, interleukin-13 (IL-13), cytokines produced by eosinophils and ILC2s, can result in a delay of recovery from CDI, suggesting that these cytokines are involved in directing downstream signaling important for resolution. With IL-4 and IL-13 being important for alternatively-activated macrophage (AAM) polarization, as well as the presence of AAM markers, it appears that this cell type can be a factor in the recovery from CDI.

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. DEFINITIONS

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “a therapeutic agent” refers to one or more therapeutic agents, e.g., one or more of the same of different therapeutic agents. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of time, concentration percent inhibition, percent viability, amounts of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” unless stated otherwise.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin subunit molecules. The antibodies in the presently disclosed subject matter can exist in a variety of forms including, for example, polyclonal antibodies, monoclonal antibodies, Fv, Fab and F(ab)₂, as well as single chain antibodies and humanized antibodies.

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “secondary antibody” refers to an antibody that binds to the constant region of another antibody (the primary antibody).

The term “antigen” as used herein is defined as a molecule that provokes an immune response. This immune response can involve either antibody production, or the activation of specific immunologically-competent cells, or both. An antigen can be derived from organisms, subunits of proteins/antigens, killed or inactivated whole cells or lysates.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen can induce the production of antibodies that bind specifically to a given region or three-dimensional structure on the protein; these regions or structures are referred to as antigenic determinants. An antigenic determinant can compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of the presently disclosed subject matter, and is effective in killing or substantially inhibiting the growth of microbes. “Antimicrobial” as used herein, includes antibacterial, antifungal, and antiviral agents.

A pathology or symptom “associated” with C. difficile infection refers to mortality, colonic inflammation, diarrhea, weight loss, changes in expression and levels of genes, proteins, and cells as described herein or those that are known in the art that occur upon the infection or are a result of the infection.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

As used herein, the term “conservative amino acid substitution” is defined herein as an amino acid exchange within one of the following five groups: I) Small aliphatic, nonpolar or slightly polar residues: Ala, Ser, Thr, Pro, Gly; II) Polar, negatively charged residues and their amides: Asp, Asn, Glu, Gin; III) Polar, positively charged residues: His, Arg, Lys; IV) Large, aliphatic, nonpolar residues: Met Leu, Ile, Val, Cys; and V) Large, aromatic residues: Phe, Tyr, Trp.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets, and effector activities of these cytokines have been described.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

“Co-administer” can include simultaneous and/or sequential administration of two or more agents.

A “compound,” as used herein, refers to any type of substance or agent that is can be considered a drug, or a candidate for use as a drug, as well as combinations and mixtures of the above.

A “control” cell, tissue, sample, or subject is a cell, tissue, sample, or subject of the same type as a test cell, tissue, sample, or subject. The control can, for example, be examined at precisely or nearly the same time the test cell, tissue, sample, or subject is examined. The control can also, for example, be examined at a time distant from the time at which the test cell, tissue, sample, or subject is examined, and the results of the examination of the control can be recorded so that the recorded results can be compared with results obtained by examination of a test cell, tissue, sample, or subject. The control can also be obtained from another source or similar source other than the test group or a test subject, where the test sample is obtained from a subject suspected of having a condition, disease, or disorder for which the test is being performed.

A “test” cell is a cell being examined.

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, a “derivative” of a compound refers to a chemical compound that can be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, the terms “condition”, “disease condition”, “disease”, “disease state”, and “disorder” refer to physiological states in which diseased cells or cells of interest can be targeted with the compositions of the presently disclosed subject matter.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, can be different from when that compound is administered alone. Thus, an effective amount of a combination of compounds refers collectively to the combination as a whole, although the actual amounts of each compound can vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment” or “segment” or “subsequence” is a portion of an amino acid sequence comprising at least one amino acid or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, can ordinarily be at least about 20 nucleotides in length, typically, at least about 50 nucleotides, more typically, from about 50 to about 100 nucleotides, at least about 100 to about 200 nucleotides, at least about 200 nucleotides to about 300 nucleotides, at least about 300 to about 350, at least about 350 nucleotides to about 500 nucleotides, at least about 500 to about 600, at least about 600 nucleotides to about 620 nucleotides, at least about 620 to about 650, and or the nucleic acid fragment will be greater than about 650 nucleotides in length.

As used herein, a “functional” biological molecule is a biological molecule in a form in which it exhibits a property by which it can be characterized. A functional enzyme, for example, is one that exhibits the characteristic catalytic activity by which the enzyme can be characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm incorporated into the NBLAST and XBLAST programs that can be accessed, for example, at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized. Alternatively, PSI-Blast or PHI-Blast can be used to perform an iterated search which detects distant relationships between molecules and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein “injecting”, “applying”, and “administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

The terms “intestinal microbiota”, “gut flora”, and “gastrointestinal microbiota” are used interchangeably to refer to bacteria in the digestive tract.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample can of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

In some embodiments, a “preventive” or “prophylactic” treatment is a treatment administered to a subject who does not exhibit signs, or exhibits only early signs, of a condition, disease, or disorder. Thus, a prophylactic or preventative treatment can be administered for the purpose of decreasing the risk of developing pathology associated with developing the condition, disease, or disorder.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

The term “reduces recurrent infection” means that the number or percentage of subjects who get another C. difficile infection following a low dose or short-term course of treatment for an initial C. difficile infection is lower compared to the number who had received standard doses or standard duration therapies.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Ayes (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, a “subject in need thereof” is a patient, animal, mammal, or human, who will benefit from the method of the presently disclosed subject matter.

The term “substantially pure” describes a compound, e.g., a protein or polypeptide, cell or nucleic acid that has been separated from components which naturally accompany it. Typically, a compound is substantially pure when at least 10%, including at least 20%, at least 50%, at least 60%, at least 75%, at least 90%, at least 95%, at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

As used herein, a “substantially homologous amino acid sequences” or “substantially identical amino acid sequences” includes those amino acid sequences which have at least about 92%, or at least about 95% homology or identity, including at least about 96% homology or identity, including at least about 97% homology or identity, including at least about 98% homology or identity, and at least about 99% or more homology or identity to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” or “substantially identical nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In one embodiment, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 92%, 95%, 99% or more. Substantial identity of nucleic acid sequences can be determined by comparing the sequence identity of two sequences, for example by physical/chemical methods (i.e., hybridization) or by sequence alignment via computer algorithm.

Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2×standard saline citrate (SSC), 0.1% SDS at 50° C.; preferably in 7% (SDS), 0.5 M NaPO₄, 1 mM EDTA at 50° C., with washing in 1×SSC, 0.1% SDS at 50° C.; preferably 7% SDS, 0.5 M NaPO₄, 1 mM EDTA at 50° C. with washing in 0.5 SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package. The default settings provided with these programs are suitable for determining substantial similarity of nucleic acid sequences for purposes of the presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented. The term “treating” refers any effect, e.g., lessening, reducing, modulating, ameliorating, reversing or eliminating, that results in the improvement of the condition, disease, disorder, and the like, or ameliorating a symptom thereof.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### II. EXEMPLARY METHODS AND COMPOSITIONS

C. difficile Infection (CDI) is the leading hospital-acquired gastrointestinal infections and contributes to a significant burden in the healthcare industry. While the bacterial toxins and their contributions to pathogenesis have been studied extensively, the impact of the robust inflammatory immune response to disease is not well understood.

The presently disclosed subject matter is based, at least in part, on the finding of beneficial effects of type 2 immunity during CDI. The cytokines IL-25 and IL-33 are protective through eosinophils and Type 2 Innate Lymphoid Cells (ILC2s), respectfully. While these upstream responses have been observed, the downstream mechanisms have not been previously determined. ILC2s produce a lot of IL-13, an important cytokine for promoting epithelial remodeling, alternative-activation of macrophages, and eosinophil recruitment. As described hereinbelow, neutralization of IL-13 leads to a delay in recovery from disease, while addition of this cytokine increases the speed of recovery, suggesting that it can have an impact in promoting disease resolution. Additionally, neutralization leads to an increase in Ly6C ^{high} monocytes at a later time point during disease, supporting the hypothesis that IL-13-mediated resolution of inflammation can have a role for promoting recovery.

The neutralization of the decoy receptor of IL-13, IL-13Ra2, has been shown to promote recovery from DSS colitis, and its expression increases during the recovery period of CDI. In at least one aspect, the presently disclosed subject matter is based on the finding that this receptor can impede endogenous IL-13 protective responses in CDI. Therefore, neutralization of this receptor can serve as a therapeutic approach for promoting recovery from this disease. Understanding the role of type 2 immunity and IL-13 during CDI can enhance the ability to utilize nonantibiotic therapy approaches to promote protection in patients.

More particularly, in one aspect, the presently disclosed subject matter relates to the decoy receptor for IL-13 being a pathogenic factor in CDI. IL-13 can limit type 1 and type 17 immunity, both inflammatory drivers of disease in CDI. Conversely, these inflammatory responses can increase IL-13Ra2 levels to impede type 2 immunity, thereby potentially inhibiting the protective functions of IL-13. The beneficial response of type 2 immunity during CDI is only beginning to be appreciated, and no known role for IL-13 has been described before. Additionally, much of the biology and functions of IL-13Ra2 have not been previously elucidated. Therefore, defining its role during CDI not only increases the understanding of the host response to C. difficile, but also of the role of this decoy receptor during immune responses.

In some embodiments, the presently disclosed subject matter relates to the administration of exogenous IL-13 or a biologically active fragment thereof and/or of exogenous IL-4 or a biologically active fragment thereof. In some embodiments, the presently disclosed subject matter relates to the use of neutralizing antibodies against a receptor (IL-13Ra2) that normally serves to block the signaling of its ligand (IL-13). Neutralization of this receptor can result in increased signaling of IL-13, without the need to administer any exogenous cytokine. Additionally, antibiotics are the first line of defense against C. difficile, but their use contributes to antibiotic resistance and increased microbiome disturbance. It is also possible that antibiotics do not address host damage and inflammatory responses. The use of immunotherapy in addition to, or in place of, traditional antibiotic treatment can increase therapeutic responses by limiting pathogenic inflammation caused by the bacteria.

Accordingly, in some embodiments, the presently disclosed subject matter provides a method of treating or preventing a C. difficile infection, or a pathology or symptom associated therewith, in a subject in need thereof by administering to said subject a composition comprising a therapeutically effective amount of at least one agent that enhances a biological activity of interleukin-13 (IL-13). For example, the agent can stimulate or increase the expression, levels, or activity of IL-13 as described herein for treating CDI. In some embodiments, the C. difficile infection is a recurring C. difficile infection (i.e., a recurrence of a C. difficile infection in a subject who has already recovered from, at least partly recovered from, or has been treated for a prior C. difficile infection).

In some embodiments, the agent that enhances a biological activity of IL-13 comprises or consists of an exogenous IL-13 peptide or a biologically active fragment or homolog thereof. Thus, in some embodiments, the method comprises administering to a subject an effective amount of an IL-13 peptide, or a biologically active fragment or homolog thereof. In some embodiments, the IL-13 peptide is a human IL-13 peptide or a mouse IL-13 peptide. Non-human and non-murine IL-13 peptides are also encompassed by the presently disclosed subject matter, as are recombinant IL-13 (rIL-13) peptides.

Exemplary useful IL-13 peptides include the following:

mhpllnplll alglmalllt tvialtclgg faspgpvpps talrelieel vnitqnqkap lcngsmvwsi nitagmycaa leslinvsgc saiektqrml sgfcphkvsa gqfsslhvrd tkievaqfvk dlllhlkklf regqfn (SEQ ID NO: 1, interleukin-13 isoform a precursor [homo sapiens], NCBI Reference Sequence: NP_002179, 146 amino acids); mvwsinitag mycaalesli nvsgcsaiek tqrmlsgfcp hkvsagqfss lhvrdtkiev aqfvkdlllh lkklfregqf n (SEQ ID NO: 2, interleukin-13 isoform b [homo sapiens], NCBI Reference Sequences: NP_001341920, NP_001341921, NP_001341922, 81 amino acids); and

malwvtavla laclgglaap gpvprsyslp ltlkelieel snitqdqtpl cngsmvwsvd laaggfcval dsltnisncn aiyrtqrilh glcnrkaptt vsslpdtkie vahfitklls ytkqlfrhgp f (SEQ ID NO: 3, interleukin-13 precursor [Mus musculus], NCBI Reference Sequence: NP_032381, 131 amino acids).

Additional useful IL-13 peptides include those described in Table 1, below.

TABLE 1
Additional Exemplary IL-13 Peptides

| GENBANK ® | | |
|---|---|---|
| Accession No. | Description | Species |
| XP_002815912.1 | interleukin-13 isoform X1 | Pongo abelii |
| XP_003829348.2 | interleukin-13 isoform X1 | Pan paniscus |
| XP_003829349.1 | interleukin-13 isoform X2 | Pan paniscus |
| NP_001008992.1 | Interleukin-13 precursor | Pan troglodytes |
| XP_016800435.1 | Interleukin-13 isoform X1 | Pan troglodytes |
| XP_004042524.1 | Interleukin-13 isoform X1 | Gorilla gorilla gorilla |
| XP_018883794.1 | Interleukin-13 isoform X2 | Gorilla gorilla gorilla |
| NP_001028101.1 | Interleukin-13 precursor | Macaca mulatta |
| XP_028704855.1 | Interleukin-13 isoform X1 | Macaca mulatta |
| XP_028704856.1 | Interleukin-13 isoform X2 | Macaca mulatta |
| NP_776514.1 | Interluekin-13 precursor | Bos taurus |
| NP_998968.1 | Interleukin-13 precursor | Sus scrofa |
| XP_020935977.1 | Interleukin-13 isoform X1 | Sus scrofa |
| XP_020935987.1 | Interleukin-13 isoform X2 | Sus scrofa |
| XP_005661700.1 | Interleukin-13 isoform X3 | Sus scrofa |
| NP_001003384.1 | Interleukin-13 precursor | Canis lupus familiaris |
| XP_022280614.1 | Interleukin-13 isoform X1 | Canis lupus familiaris |
| XP_022280615.1 | Interleukin-13 isoform X2 | Canis lupus familiaris |
| XP_006927648.2 | Interleukin-13 isoform X1 | Felis catus |
| XP_003980929.3 | Interleukin-13 isoform X2 | Felis catus |
| XP_006927649.2 | Interleukin-13 isoform X3 | Felis catus |
| NP_001076063.1 | Interleukin-13 precursor | Ovis aries |
| NP_001137263.1 | Interleukin-13 precursor | Equus caballus |
| NP_446280.1 | Interleukin-13 precursor | Rattus norvegicus] |
| XP_032767543.1 | Interleukin-13 | Rattus rattus |
| NP_001007086.1 | Interleukin-13 isoform X1 | Gallus gallus |
| NP_001182720.1 | Interleukin-13 isoform X2 | Gallus gallus |

Thus, in some embodiments, the IL-13 peptide comprises or consists of an amino acid sequence selected from the group including SEQ ID NOs: 1, 2, and 3 and the peptides referenced in Table 1, above, as well as biologically active fragments and homologs thereof.

The presently disclosed subject matter encompasses the use of active isoforms of IL-13 peptides. Fragments of IL-13 peptides can include one or more amino acid residue deletions (e.g., up to 5, up to 10, up to 15, up to 20, up to 25, up to 30, up to 35, up to 40, up to 50, up to 60, or up to 70 amino acid deletions), while homologs can comprise peptides having amino acid sequences that are homologous to an IL-13 peptide sequence described herein.

In some embodiments, the presently disclosed subject matter provides for the use of IL-13 peptides with conservative amino acid substitutions that do not have a substantial effect on the activity described herein. For example, there can be up to 20 or up to about 10 conservative amino acid substitutions. In one aspect, there are 1, 2, 3, 4, 5, 6, 7, 8, 9, or 10 substitutions.

Useful homologs of IL-13 peptides and its biologically active fragments can have varying homologies as long as the homologous peptide has similar activity. In one aspect, the homology can be at least about 75, 80, 85, 90, 95, and 99% homology with the IL-13 peptide or fragment. In some embodiments, the homology is at least about 95%, 96%, 97%, 98%, or 99%. In some embodiments, the IL-13 peptide has an amino acid sequence that is at least 90% or 95% identical to one of SEQ ID NO: 1, SEQ ID NO: 2; or SEQ ID NO: 3. In some embodiments, the IL-13 peptide comprises or consists of an amino acid sequence that is at least 90% or 95% identical to a peptide of Table 1.

Amino acids 1-20 of SEQ ID NO: 1 and amino acids 1-28 of SEQ ID NO: 3 correspond to signal sequences. Accordingly, in some embodiments, the IL-13 peptide comprises or consists of an amino acid sequence that is 90% or 95% identical to the sequence consisting of amino acids 21-146 of SEQ ID NO: 1 or amino acids 19-131 of SEQ ID NO: 3.

Effective doses of IL-13 peptide can vary depending on the age, sex, weight, and health of the subject and a dosage regimen or strategy can be developed by one of ordinary skill in the art. In one embodiment, an effective dose of IL-13 peptide or a biologically active fragment or homolog thereof ranges from about 0.1 μg/kg body weight to about 1,000 μg/kg body weight. In one aspect, the dosage is from about 1.0 μg/kg body weight to about 500 μg/kg body weight. In another aspect, the dosage is from about 5.0 μg/kg body weight to about 200 μg/kg body weight. In a further aspect, the dosage is from about 10 μg/kg body weight to about 100 μg/kg body weight. In another aspect, the dosage is selected from the group consisting of 1.0, 2.0, 3.0, 4.0, 5.0, 6.0, 7.0, 8.0, 9.0, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 350, 375, 400, 425, 450, 475, and 500 μg/kg of body weight.

Clinical doses of proteins or biologically active fragments or homologs thereof are disclosed herein and are also known in the art. Doses can vary depending on the age, sex, weight, body surface area (BSA), and health of the subject, as well as the specific sign(s) of the infection being treated. Additionally, a higher dose can be used in some cases where an immediate effect is needed. Doses can be administered as a unit dose or the dose can be based on criteria such as those described above for body weight, etc. Doses can also be divided if administered more than once per day.

The number of doses of IL-13 peptide to be administered can also vary depending on the age, sex, weight, body surface area (BSA), and health of the subject, as well as the specific sign(s) of the infection being treated. A pharmaceutical composition of the presently disclosed subject matter can be administered, for example, 1, 2, 3, 4, 5, 6, 7, 8, 9, or 10 times. In one aspect, it can be administered up to 20 times. In one aspect, it is administered once daily. In another aspect it is administered more than once in a day. In one aspect, it is administered once per week. In another aspect, it is administered more than once per week. In one aspect, it is administered once per month. In another aspect, it is administered more than once per month.

In some embodiments, the agent that enhances a biological activity of IL-13 is an agent that blocks or neutralizes IL-13Ra2. The agent that blocks or neutralizes IL-13Ra2 can comprise, without limitation, a drug, a small molecule (e.g., a natural or synthetic compound with a molecular weight of less than about 900 Daltons (Da), less than about 800 Da, less than about 700 Da or less than about 600 Da), an antibody, an antigen binding portion thereof or a biosynthetic antibody binding site that binds a particular target protein; an antisense molecule that hybridizes in vivo to a nucleic acid encoding IL13Ra2 (e.g., human IL-13Ra2 DNA (GENBANK® Accession No. NG_012514) or mRNA (GENBANK® Accession No. NM_000640)) or a fragment or regulatory element associated therewith, or a ribozyme, aptamer, or small molecule that binds to and/or inhibits IL-13Ra2 or that binds to and/or inhibits, reduces or otherwise modulates expression of nucleic acid encoding IL-13Ra2. In some embodiments, the blocking/neutralizing agent is an anti-IL-13Ra2 antibody (e.g., an anti-IL-13Ra2 monoclonal antibody or an anti-IL-13Ra2 polyclonal antibody, or a fragment thereof). In some embodiments, the anti-I L-13Ra2 antibody is a monoclonal antibody.

Anti-IL-13Ra2 antibodies are known in the art. The antibodies can be elicited via techniques known in the art using antigens comprising full length human IL-13Ra2 (Uniprot No. Q14627; NP_000631) or a fragment thereof (e.g., the fragment comprising amino acids 43-330). Commercially available monoclonal and polyclonal anti-IL-13Ra2 antibodies include the antibodies with catalog numbers AB55275, AB74730, and AB108534 from Abcam (Cambridge, United Kingdom), catalog numbers 130-104-670, 130-104-598, or 120-104-553 from Miltenyi Biotech (Bergisch Gladbach, Germany), catalog numbers 614 and 6141 from R&D Systems (Minneapolis, Minnesota, United States of America), catalog numbers SC-57267, and SC-74160 from Santa Cruz Biotechnology (Santa Cruz, Calif., United States of America), catalog numbers H00003598-M01, H00003598-B01P, and H00003598-D01 from Abnova (Taipei City, Taiwan), catalog numbers ALX-804-506-C100 from Enzo Life Sciences (Ann Arbor, Mich., United States of America) and catalog numbers AM31179AFN, AM31179RP-N, and AM31180AF-N from Origene (Rockville, Md., United States of America). In addition, antibodies against IL-13Ra2 (and their related antigenic determinants) have been described in U.S. Patent Application Publication Nos. 2019/0359723 and 2020/0299394, the disclosures of each of which are incorporated by reference in their entirety.

When used in vivo for therapy, the antibodies of the presently disclosed subject matter are administered to the subject in a therapeutically effective amount (i.e., an amount that has a desired therapeutic effect). Typically, the antibodies can be administered parenterally. The dose and dosage regimen can depend upon the degree of the infection, the characteristics of the particular antibody used, e.g., its therapeutic index, the patient, and the patient's history. In some embodiments, the antibody is administered continuously over a period of 1-2 weeks.

In some embodiments, a therapeutic dose of an antibody of the presently disclosed subject matter, including, but not limited to, monoclonal antibodies, chimeric antibodies, humanized antibodies, various kinds of fragments, and biologically active homologs and fragments thereof, is from about 0.1 mg/dose to about 5,000 mg/dose or from about 0.2 mg/dose to about 1,000 mg/dose. Doses can also be administered based on body weight, for example at a dosage ranging from about 0.01 mg/kg to about 1,000 mg/kg body weight or from about 0.1 to about 500 mg/kg.

In some embodiments, an effective dose of the agent that enhances a biological activity of IL-13 is one that is sufficient to treat infection and/or control diarrhea and/or weight loss in a subject infected with C. difficile. Thus, in some embodiments, the administering inhibits diarrhea e.g., eliminates or reduces diarrhea compared to what would be expected in the absence of the administering. In some embodiments, the administering results inhibits weight loss, e.g., eliminates or reduces weight loss compared to what would be expected in the absence of the administering. In some embodiments, the administering inhibits colonic inflammation, e.g., eliminates or reduces colonic inflammation compared to what would be expected in the absence of the administering or the level of inflammation prior to the administering. In some embodiments, intestinal gut flora is preserved (e.g., such that recurrent disease is prevented).

In some embodiments, an effective dose is a dose that reduces mortality.

In some embodiments, the compositions and methods of the presently disclosed subject matter are useful for preventing relapse in an already treated subject and in preventing reinfection, as well as reducing the frequency of relapse or reinfection.

In some embodiments, the subject in need of treatment is a mammal. In some embodiments, the subject is a human. In some embodiments, the subject is a subject who has a C. difficile infection or a subject who has one or more increased risk factors for a C. difficile infection. Increased risk factors for getting a C. difficile infection include, but are not limited to, hospitalization (current or recent hospitalization, such as hospitalization in the last one or two weeks), antibiotic use (current or recent antibiotic use), and increased age. For example, humans aged 45 years or older have higher risk for CDI than younger adult humans, and risk continues to increase with age. Thus, for example, the subject with increased risk can be a human that is over 45 years of age, over 55 years of age, or over 65 years of age.

In some embodiments, the administration is made during the course of adjunct therapy, such as antimicrobial treatment, or administration of a tumor necrosis factor, interferon, or other cytoprotective or immunomodulatory agent. Thus, in some embodiments, the composition of the presently disclosed subject matter can further comprise at least one additional therapeutic agent, alone or in combination (e.g., 2, 3, or 4 additional therapeutic agents). In some embodiments, one or more additional therapeutic agent can be administered as part of one or more separate compositions from that comprising the agent that enhances a biological activity of IL-13 (e.g., an IL-13 peptide or an anti-IL-13Ra2 antibody). Examples of additional therapeutic agents for use according to the presently disclosed subject matter include, but are not limited to: (a) antimicrobials, (b) steroids (e.g., hydrocortisone, triamcinolone); (c) analgesics and/or anesthetics (e.g., aspirin, an NSAID, a local anesthetic); (d) anti-inflammatory agents; (e) growth factors, (f) cytokines other than IL-13, and (g) combinations thereof.

In some embodiments, the at least one additional therapeutic agent comprises a cytokine. Any suitable cytokine can be used, such as, but not limited to, a growth factor, an interferon (IFN-α, IFN-γ), an interleukin (e.g., IL-25, IL-33, IL-4, etc.), a tumor necrosis factor (TNF-α), a lymphokine, a chemokine, etc. In some embodiments, the at least one additional therapeutic agent comprises an interleukin. In some embodiments, the at least one additional therapeutic agent comprises IL-4. In some embodiments, the at least one additional therapeutic agent comprises IL-25 or IL-33.

One of ordinary skill in the art will appreciate that, the dose of an IL (e.g., IL-33 or IL-25) (or biologically active fragments and homologs thereof) can be varied depending on such things as the age, health, sex, and age of the subject as well as the severity of the CDI or whether it is being used as a preventative. For example, mice can receive 0.5 μg to 1.25 μg of recombinant IL-33 (IL-33) intraperitoneally daily for 5 days prior to infection or for various amounts of time. Considering that mice can range from 20 to 30 grams, calculations for ranges and unit doses can be extrapolated to humans. For example, if a mouse is approximately 20 grams in weight, when this dose is translated to humans, the dose would be approximately 25 μg of rIL-33/kg body weight to about 62.5 μg of rIL-33/kg body weight. Additionally, depending on various parameters regarding the subject, whether a dose is provided in one administration to a subject or as multiples, the presently disclosed subject matter further encompasses doses of about 1.0 μg/kg body weight to about 500 μg/kg body weight. In one aspect, the range is about 2.0 to about 150 μg IL/kg body weight. In another aspect, the range is about 5.0 to about 100 μg/kg body weight. In yet another aspect, the dose range is about 10 to about 75 μg/kilo body weight. In a further aspect, the range is about 20 to about 50 μg/kg body weight. The doses include fractions and decimals of the doses provided herein. In one aspect, the therapeutically effective dose used is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 58, 59, 60, 61, 62, 62.5, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 μg IL/kg body weight, and decimals thereof. The total amount to be administered during a day can be divided into lower doses and administered at multiple times/day. In one aspect, the method is useful for low dose treatment.

Antimicrobial agents include, but are not limited to, antibacterial, antifungal, and antiviral agents. For example, antimicrobial agents include silver sulfadiazine, nystatin, nystatin/triamcinolone, bacitracin, nitrofurazone, nitrofurantoin, a polymyxin, doxycycline, antimicrobial peptides, beosporin, polysporin, silver salts, iodine, benzalkonium chloride, alcohol, hydrogen peroxide, and chlorhexidine. Additional examples of antimicrobial agents include, but are not limited to, isoniazid, ethambutol, pyrazinamide, streptomycin, clofazimine, rifabutin, fluoroquinolones, ofloxacin, sparfloxacin, rifampin, rifaximin, azithromycin, clarithromycin, dapsone, tetracycline, erythromycin, cikprofloxacin, doxycycline, ampicillin, amphotericine B, ketoconazole, fluconazole, pyrimethamine, sulfadiazine, clindamycin, lincomycin, pentamidine, atovaquone, paromomycin, diclarazaril, acyclovir, vancomycin, fidaxomicin, metronidazole, nitazoxanide, trifluorouridine, foscarnet, penicillin, gentamicin, ganciclovir, iatroconazole, miconazole, Zn-pyrithione, and silver salts, such as chloride, bromide, iodide, and periodate. In some embodiments, the antimicrobial agent comprises at least one antibiotic selected from the group comprising vancomycin, fidaxomicin, metronidazole, nitazoxanide, and rifaximin.

In some embodiments, for short durations of treatment the presently disclosed subject matter provides a dosage range of at least one antibiotic of about 0.1 mg/kg to about 75 mg/kg. In some embodiments, it is from about 0.5 to about 50 mg/kg. In some embodiments, it is from about 2.0 to about 40 mg/kg. In some embodiments, it is from about 3.0 to about 35 mg/kg. In some embodiments, it is from about 4.0 to about 30 mg/kg. In a further aspect, it is from about 5.0 to about 25 mg/kg. In some embodiments, it is from about 6.0 to about 20 mg/kg. In some embodiments, it is from about 7.0 to about 15 mg/kg. In some embodiments, the dose is about 0.1, 0.5, 1.0, 2.0, 3.0, 4.0, 5.0, 6.0, 7.0, 8.0, 9.0, 10, 15, 20, 25, 30, 32, 35, 40, 45, 55, 60, 65, 70, or about 75 mg/kg. One of ordinary skill in the art can determine which dose to use depending on whether the treatment is for a short duration, or for a low dose, or a combination of the two. In some embodiments, a short-term treatment such as 1 or 2 days, can use a slightly higher dose than a treatment that lasts longer.

The total amount to be administered during a day can be divided into lower doses and administered at multiple times/day. In some embodiments, the method is useful for low dose treatment. In some embodiments, the method is useful for short-term treatment. For example, if 20 mg/kg/day is the prescribed amount for the day, that amount can be divided into more than one dose for administration during the day, such as doses of 10 mg/kg administered twice. In some embodiments, treatment can be as short as 1 day. In some embodiments, even doses as low as 0.01, 0.1, 0.5, 1.0, 1.5, 2.0, 2.5, 3.0, 3.5, 4.0, 4.5, and 5.0 mg/kg/day can be administered as partial doses multiple times in a day when it is determined that the entire daily dose does not need to be administered in one bolus or that it would be better to administer the daily dose in several increments.

One of ordinary skill in the art can determine the best route of administration of a pharmaceutical composition of the presently disclosed subject matter. For example, administration can be direct, enteral, or parenteral. Enteral includes, for example, oral and rectal administration. Parenteral includes, for example, intravenous administration. One of ordinary skill in the can determine the method and site of administration. For example, enteral, parental, direct, intravenous, or subcutaneous injection a composition comprising a protein agent (or biologically active fragments or homologs thereof), such as an antibody or cytokine, can be an effective treatment.

A compound or composition of the presently disclosed subject matter can be administered once or more than once. It can be administered once a day or at least twice a day. In one aspect, a compound is administered every other day within a chosen term of treatment. In one embodiment, at least two compounds of the presently disclosed subject matter are used. One of ordinary skill in the art can determine how often to administer a compound of the presently disclosed subject matter, the duration of treatment, and the dosage to be used.

In some embodiments, the present disclosed subject matter provides a compositions and methods involving the use of probiotics, prebiotics, or narrow spectrum antibiotics/anti-bacterial agents that are capable of restoring healthy mammalian bacterial gastrointestinal microbiota. The composition of the presently disclosed subject matter can further include growth factors, nutrient factors, pharmaceuticals, calcium-containing compounds, anti-inflammatory agents, antimicrobial agents, or any other substance capable of expediting or facilitating the treatment.

When two or more compounds are to be administered, they can be administered in the same pharmaceutical composition or in separate pharmaceutical compositions. When administered in separate pharmaceutical compositions, they can be administered simultaneously or one can be administered first. The amount of time between administration of the different compounds can vary and can be determined by one of ordinary skill in the art. For example, the two compounds could be administered up to 10 minutes apart, up to 30 minutes apart, up to 1 hour apart, etc. In one aspect, one or more of the compounds can be administered more than once. In one aspect, a compound is administered at least twice. In another aspect, a compound is administered at least five times. In one aspect, the method is useful for low dose treatment. In one aspect, the method is useful for short-term treatment.

In some embodiments, duration of treatment is from about 1-10, 1-9, 1-8, 1-7, 1-6, 1-5, 1-4, 1-3, or 1-2 days. In some embodiments, duration of treatment is from about 2-10, 2-9, 2-8, 2-7, 2-6, 2-5, 2-4, or 2-3 days. In some embodiments, duration of treatment is from about 3-10, 3-9, 3-8, 3-7, 3-6, 3-5, or 3-4 days. In some embodiments, duration of treatment is from about 4-10, 4-9, 4-8, 4-7, 4-6, or 4-5 days. In some embodiments, duration of treatment is from about 5-10, 5-9, 5-8, 5-7, or 5-6 days. In some embodiments, duration of treatment is from about 6-10, 6-9, 6-8, or 6-7 days. In some embodiments, duration of treatment is from about 7-10, 7-9, or 7-8 days. In some embodiments, duration of treatment is from about 8-10 or 8-9. In some embodiments, treatment is for about 1, 2, 3, 4, 5, 6, 7, 8, 9, or 10 days.

In some embodiments, a subject is treated daily during the treatment regimen when the duration is longer than one day. In another aspect, the subject is treated every other day.

In some embodiments, the presently disclosed subject matter provides a composition for use in treating or preventing a CDI, or a symptom or pathology associated therewith, wherein the composition comprises at least one agent that enhances a biological activity of IL-13. In some embodiments, the agent comprises or consists of an IL-13 peptide or fragment of homolog thereof. In some embodiments, the agent comprises or consists of an IL-13 peptide that has an amino acid sequence that is at least 75%, 80%, 85%, 90%, or 95% identical to one of SEQ ID NO: 1, SEQ ID NO: 2, and SEQ ID NO: 3. In some embodiments, the agent comprises or consists of an agent that blocks or neutralizes IL-13Ra2. In some embodiments, the agent comprises or consists of an anti-IL-13Ra2 antibody. In some embodiments, the anti-IL-13Ra2 antibody is a monoclonal antibody.

In some embodiments, the composition further comprises at least one additional therapeutic agent, e.g., an anesthetic, an analgesic, an antimicrobial, a steroid, a growth factor, a cytokine other than IL-13, an anti-inflammatory agent, or a combination thereof. In some embodiments, the composition is for use in treating a subject who has a CDI. In some embodiments, the composition is for use in treating a subject with one or more increased risk factors fora CDI. In some embodiments, the composition is for use in treating or preventing recurring CDI.

In some embodiments, the presently disclosed subject matter provides a method of treating or preventing a C. difficile infection, or a pathology or symptom associated therewith, in a subject in need thereof by administering to said subject a composition comprising a therapeutically effective amount of a interleukin-4 (IL-4) peptide or a biologically active fragment or homolog thereof. In some embodiments, the C. difficile infection is a recurring C. difficile infection (i.e., a recurrence of a C. difficile infection in a subject who has already recovered from, at least partly recovered from, or has been treated for a prior C. difficile infection).

Thus, in some embodiments, the method comprises administering to a subject an effective amount of an I L-4 peptide, or a biologically active fragment or homolog thereof. In some embodiments, the IL-4 peptide is a human IL-4 peptide or a mouse IL-4 peptide. Non-human and non-murine IL-4 peptides are also encompassed by the presently disclosed subject matter, as are recombinant IL-4 (rIL-4) peptides.

Exemplary useful IL-4 peptides include the following:

mgltsqllpp lffllacagn fvhghkcdit lqeiiktlns lteqktlcte ltvtdifaas kntteketfc raatvlrqfy shhekdtrcl gataqqfhrh kqlirflkrl drnlwglagl nscpvkeanq stlenflerl ktimrekysk css (SEQ ID NO: 4, interleukin-4 isoform 1 precursor [homo sapiens], NCBI Reference Sequence: NP_000580, 153 amino acids);

mgltsqllpp lffllacagn fvhghkcdit lqeiiktlns lteqknttek etfcraatvl rqfyshhekd trclgataqq fhrhkqlirf lkrldrnlwg laglnscpvk eanqstlenf lerlktimre kyskcss (SEQ ID NO: 5, interleukin-4 isoform 2 precursor [homo sapiens], NCBI Reference Sequence: NP_758858, 137 amino acids);

mgltsqllpp lffllacagn fvhghkcdit lqeiiktlns lteqktlcte ltvtdifaas kqhaclptsr htgticrpps arvpststrl qspertqlrr kpsaglrlcs gsstatmrrt laawvrlhss stgtss (SEQ ID NO: 6, interleukin-4 isoform 3 precursor [Homo sapiens], NCBI Reference Sequence NP 001341919, 136 amino acids); and

mglnpqlwi llfflectrs hihgcdknhl reiigilnev tgegtpctem dvpnvltatk ntteselvcr askvlrifyl khgktpclkk nssvlmelqr lfrafrclds sisctmnes stslkdfles lksimqmdys (SEQ ID NO: 7. Interleukin-4 precursor [mus musculus], NCBI Reference Sequence: NP_067258, 140 amino acids).

Additional useful IL-4 peptides include those described in Table 2, below.

TABLE 2
Additional Exemplary IL-4 Peptides

| GENBANK ® Acc. No. | Description | Species |
|---|---|---|
| NP_776346.1 | interleukin-4 precursor | Bos taurus |
| NP_001003159.1 | interleukin-4 precursor | Canis lupus familiaris |
| NP_001075988.1 | interleukin-4 precursor | Equus caballus |
| NP_001036804.1 | interleukin-4 precursor | Felis catus |
| XP_004042527.1 | interleukin-4 isoform X1 | Gorilla gorilla gorilla |
| XP_004042528.1 | interleukin-4 isoform X2 | Gorilla gorilla gorilla |
| NP_001028076.1 | interleukin-4 precursor | Macaca mulatta |
| XP_011714931.1 | interleukin-4 isoform X2 | Macaca nemestrina |
| XP_011824653.1 | interleukin-4 isoform X2 | Mandrillus leucophaeus |
| NP_001009313.3 | interleukin-4 precursor | Ovis aries |
| XP_003829346.1 | interleukin-4 isoform X1 | Pan paniscus |
| XP_003829347.1 | interleukin-4 isoform X2 | Pan paniscus |
| NP_001011714.1 | interleukin-4 isoform 1 precursor | Pan troglodytes |
| NP_001008993.1 | interleukin-4 isoform delta2 precursor | Pan troglodytes |
| XP_007087828.1 | interleukin-4 | Panthera tigris altaica |
| NP_001106123.1 | interleukin-4 precursor | Papio anubis |
| XP_032483105.1 | interleukin-4 | Phocoena sinus |
| XP_007106325.1 | interleukin-4 | Physeter catodon |
| XP_023052181.1 | interleukin-4 | Piliocolobus tephrosceles |
| XP_036280697.1 | interleukin-4 | Pipistrellus kuhlii |
| XP_002815914.1 | interleukin-4 isoform X1 | Pongo abelii |
| XP_002815915.1 | interleukin-4 isoform X2 | Pongo abelii |
| NP_958427.1 | Interleukin-4 precursor | Rattus norvegicus |
| NP_999288.1 | interleukin-4 precursor | Sus scrofa |

Thus, in some embodiments, the IL-4 peptide comprises or consists of an amino acid sequence selected from the group including SEQ ID NOs: 4, 5, 6, and 7, and the peptides referenced in Table 2, above, as well as biologically active fragments and homologs thereof.

The presently disclosed subject matter encompasses the use of active isoforms of IL-4 peptides. Fragments of IL-4 peptides can include one or more amino acid residue deletions (e.g., up to 5, up to 10, up to 15, up to 20, up to 25, up to 30, up to 35, up to 40, up to 50, up to 60, or up to 70 amino acid deletions), while homologs can comprise peptides having amino acid sequences that are homologous to an IL-4 peptide sequence described herein.

In some embodiments, the presently disclosed subject matter provides for the use of IL-4 peptides with conservative amino acid substitutions that do not have a substantial effect on the activity described herein. For example, there can be up to 20 or up to about 10 conservative amino acid substitutions. In one aspect, there are 1, 2, 3, 4, 5, 6, 7, 8, 9, or 10 substitutions.

Useful homologs of IL-4 peptides and its biologically active fragments can have varying homologies as long as the homologous peptide has similar activity. In one aspect, the homology can be at least about 75, 80, 85, 90, 95, and 99% homology with the IL-4 peptide or fragment. In some embodiments, the homology is at least about 95%, 96%, 97%, 98%, or 99%. In some embodiments, the IL-4 peptide has an amino acid sequence that is at least 90% or 95% identical to one of SEQ ID NO: 4, SEQ ID NO: 5; SEQ ID NO: 6 or SEQ ID NO: 7. In some embodiments, the IL-4 peptide comprises or consists of an amino acid sequence that is at least 90% or 95% identical to a peptide of Table 2.

Amino acids 1-24 of SEQ ID NO: 4 and amino acids 1-20 of SEQ ID NO: 7 correspond to signal sequences. Accordingly, in some embodiments, the IL-4 peptide comprises or consists of an amino acid sequence that is 90% or 95% identical to the sequence consisting of amino acids 25-153 of SEQ ID NO: 4 or amino acids 21-140 of SEQ ID NO: 7.

Effective doses of IL-4 peptide can vary depending on the age, sex, weight, and health of the subject and a dosage regimen or strategy can be developed by one of ordinary skill in the art. In one embodiment, an effective dose of IL-4 peptide or a biologically active fragment or homolog thereof ranges from about 0.1 μg/kg body weight to about 1,000 μg/kg body weight. In one aspect, the dosage is from about 1.0 μg/kg body weight to about 500 μg/kg body weight. In another aspect, the dosage is from about 5.0 μg/kg body weight to about 200 μg/kg body weight. In a further aspect, the dosage is from about 10 μg/kg body weight to about 100 μg/kg body weight. In another aspect, the dosage is selected from the group consisting of 1.0, 2.0, 3.0, 4.0, 5.0, 6.0, 7.0, 8.0, 9.0, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 350, 375, 400, 425, 450, 475, and 500 μg/kg of body weight.

Clinical doses of proteins or biologically active fragments or homologs thereof are disclosed herein and are also known in the art. Doses can vary depending on the age, sex, weight, body surface area (BSA), and health of the subject, as well as the specific sign(s) of the infection being treated. Additionally, a higher dose can be used in some cases where an immediate effect is needed. Doses can be administered as a unit dose or the dose can be based on criteria such as those described above for body weight, etc. Doses can also be divided if administered more than once per day.

The number of doses of IL-4 peptide to be administered can also vary depending on the age, sex, weight, body surface area (BSA), and health of the subject, as well as the specific sign(s) of the infection being treated. A pharmaceutical composition of the presently disclosed subject matter can be administered, for example, 1, 2, 3, 4, 5, 6, 7, 8, 9, or 10 times. In one aspect, it can be administered up to 20 times. In one aspect, it is administered once daily. In another aspect it is administered more than once in a day. In one aspect, it is administered once per week. In another aspect, it is administered more than once per week. In one aspect, it is administered once per month. In another aspect, it is administered more than once per month.

In some embodiments, an effective dose of the IL-4 peptide or biologically active fragment or homolog thereof is one that is sufficient to treat infection and/or control diarrhea and/or weight loss in a subject infected with C. difficile. Thus, in some embodiments, the administering inhibits diarrhea e.g., eliminates or reduces diarrhea compared to what would be expected in the absence of the administering. In some embodiments, the administering results inhibits weight loss, e.g., eliminates or reduces weight loss compared to what would be expected in the absence of the administering. In some embodiments, the administering inhibits colonic inflammation, e.g., eliminates or reduces colonic inflammation compared to what would be expected in the absence of the administering or the level of inflammation prior to the administering. In some embodiments, intestinal gut flora is preserved (e.g., such that recurrent disease is prevented).

In some embodiments, an effective dose is a dose that reduces mortality.

In some embodiments, the compositions and methods of the presently disclosed subject matter are useful for preventing relapse in an already treated subject and in preventing reinfection, as well as reducing the frequency of relapse or reinfection.

In some embodiments, the subject in need of treatment is a mammal. In some embodiments, the subject is a human. In some embodiments, the subject is a subject who has a C. difficile infection or a subject who has one or more increased risk factors for a C. difficile infection. Increased risk factors for getting a C. difficile infection include, but are not limited to, hospitalization (current or recent hospitalization, such as hospitalization in the last one or two weeks), antibiotic use (current or recent antibiotic use), and increased age (e.g., a human over the age of 45 or over the age of 65).

In some embodiments, the administration is made during the course of adjunct therapy, such as antimicrobial treatment, or administration of a tumor necrosis factor, interferon, or other cytoprotective or immunomodulatory agent. Thus, in some embodiments, the composition of the presently disclosed subject matter can further comprise at least one additional therapeutic agent, alone or in combination (e.g., 2, 3, or 4 additional therapeutic agents). In some embodiments, one or more additional therapeutic agent can be administered as part of one or more separate compositions from that comprising the IL-4 peptide or biologically active fragment or homolog thereof. Examples of additional therapeutic agents for use according to the presently disclosed subject matter include, but are not limited to: (a) antimicrobials, (b) steroids (e.g., hydrocortisone, triamcinolone); (c) analgesics and/or anesthetics (e.g., aspirin, an NSAID, a local anesthetic); (d) anti-inflammatory agents; (e) growth factors, (f) cytokines other than IL-4, and (g) combinations thereof.

In some embodiments, the presently disclosed subject matter provides a composition for use in treating or preventing a CDI, or a symptom or pathology associated therewith, wherein the composition comprises or consists of an IL-4 peptide or fragment of homolog thereof. In some embodiments, the IL-4 peptide has an amino acid sequence that is at least 75%, 80%, 85%, 90%, or 95% identical to one of SEQ ID NO. 4, SEQ ID NO. 5, SEQ ID NO: 6 and SEQ ID NO: 7.

In some embodiments, the composition further comprises at least one additional therapeutic agent, e.g., an anesthetic, an analgesic, an antimicrobial, a steroid, a growth factor, a cytokine other than IL-4, an anti-inflammatory agent, or a combination thereof. In some embodiments, the composition is for use in treating a subject who has a CDI. In some embodiments, the composition is for use in treating a subject with one or more increased risk factors fora CDI. In some embodiments, the composition is for use in treating or preventing recurring CDI.

### III. ANTIBODIES, PEPTIDES AND PROTEINS

Antibodies directed against proteins, polypeptides, or peptide fragments thereof of the presently disclosed subject matter can be generated using methods that are well known in the art. For instance, U.S. Pat. No. 5,436,157, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants can be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

In some embodiments, antibodies or antisera, directed against IL-13Ra2 or a homolog or fragment thereof, are useful for blocking the activity of IL-13Ra2 (thereby increasing the activity of IL-13 (e.g., endogenous IL-13)).

Fragments of IL-13Ra2 can be generated and antibodies prepared against the fragments. Assays are provided herein to determine whether an antibody directed against IL-13Ra2, or a fragment thereof, have the ability to inhibit IL-13Ra2 activity.

For the preparation of monoclonal antibodies, any technique which provides for the production of antibody molecules by continuous cell lines in culture can be utilized. For example, the hybridoma technique originally developed by Kohler and Milstein (1975, Nature 256:495-497), the trioma technique, the human B-cell hybridoma technique (Kozbor et al., 1983, Immunology Today 4:72), and the EBV-hybridoma technique (Cole et al., 1985, in Monoclonal Antibodies and Cancer Therapy, Alan R. Liss, Inc., pp. 77-96) can be employed to produce human monoclonal antibodies. In some embodiments, monoclonal antibodies are produced in germ-free animals.

In some embodiments, human antibodies can be used and obtained by utilizing human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, Inc., pp. 77-96). Furthermore, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for epitopes of IL-13Ra2 polypeptides together with genes from a human antibody molecule of appropriate biological activity can be employed; such antibodies are within the scope of the presently disclosed subject matter. Once specific monoclonal antibodies have been developed, the preparation of mutants and variants thereof by conventional techniques is also available.

In some embodiments, techniques described for the production of single-chain antibodies (U.S. Pat. No. 4,946,778, incorporated by reference herein in its entirety) are adapted to produce protein-specific single-chain antibodies. In another embodiment, the techniques described for the construction of Fab expression libraries (Huse et al., 1989, Science 246:1275-1281) are utilized to allow rapid and easy identification of monoclonal Fab fragments possessing the desired specificity for specific antigens, proteins, derivatives, or analogs of the presently disclosed subject matter.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which specifically bind the antigen therefrom.

Monoclonal antibodies directed against full length or peptide fragments of a protein or peptide can be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide can also be synthesized using chemical synthesis technology. Alternatively, DNA encoding the desired peptide can be cloned and expressed from an appropriate promoter sequence in cells suitable for the generation of large quantities of peptide. Monoclonal antibodies directed against the peptide are generated from mice immunized with the peptide using standard procedures as referenced herein.

A nucleic acid encoding the monoclonal antibody obtained using the procedures described herein can be cloned and sequenced using technology which is available in the art, and is described, for example, in Wright et al. (1992, Critical Rev. in Immunol. 12(3,4):125-168) and the references cited therein. Further, the antibody of the presently disclosed subject matter can be “humanized” using the technology described in Wright et al., (supra) and in the references cited therein, and in Gu et al. (1997, Thrombosis and Nematocyst 77(4):755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage that encode the desired antibody can be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art.

Processes such as those described above have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures presented above describe the generation of phage which encode the Fab portion of an antibody molecule. However, the presently disclosed subject matter should not be construed to be limited solely to the generation of phage encoding Fab antibodies. Rather, phage which encode single chain antibodies (scFv/phage antibody libraries) are also included in the presently disclosed subject matter. Fab molecules comprise the entire Ig light chain, that is, they comprise both the variable and constant region of the light chain but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA can be generated following the procedures described in Marks et al., 1991, J. Mol. Biol. 222:581-597. Panning of phage so generated for the isolation of a desired antibody is conducted in a manner similar to that described for phage libraries comprising Fab DNA.

The presently disclosed subject matter should also be construed to include synthetic phage display libraries in which the heavy and light chain variable regions can be synthesized such that they include nearly all possible specificities (Barbas, 1995, Nature Medicine 1:837-839; de Kruif et al. 1995, J. Mol. Biol. 248:97-105).

In the production of antibodies, screening for the desired antibody can be accomplished by techniques known in the art, e.g., ELISA (enzyme-linked immunosorbent assay). Antibodies generated in accordance with the presently disclosed subject matter can include, but are not limited to, polyclonal, monoclonal, chimeric (i.e., “humanized”), and single chain (recombinant) antibodies, Fab fragments, and fragments produced by a Fab expression library.

The peptides of the presently disclosed subject matter can be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the a-amino group of the amino acid, and on any side chain functional groups. Side chain protecting groups are generally stable to the solvents, reagents and reaction conditions used throughout the synthesis, and are removable under conditions that will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method that utilized tert-butyloxcarbonyl as the a-amino protecting group, and the FMOC method which utilizes 9-fluorenylmethyloxcarbonyl to protect the a-amino of the amino acid residues, both methods of which are well-known by those of skill in the art.

To ensure that the proteins or peptides obtained from either chemical or biological synthetic techniques is the desired peptide, analysis of the peptide composition should be conducted. Such amino acid composition analysis can be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, can also be used to determine definitely the sequence of the peptide.

Prior to its use, the peptide can be purified to remove contaminants. In this regard, it will be appreciated that the peptide will be purified to meet the standards set out by the appropriate regulatory agencies. Any one of a number of a conventional purification procedures can be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure peptide obtained as described herein can be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

The presently disclosed subject matter encompasses methods of screening compounds to identify those compounds that act as agonists (stimulate) or antagonists (inhibit) of the protein interactions and pathways described herein. Screening assays for antagonist compound candidates are designed to identify compounds that bind or complex with the peptides described herein, or otherwise interfere with the interaction of the peptides with other cellular proteins. Such screening assays will include assays amenable to high-throughput screening of chemical libraries, making them particularly suitable for identifying small molecule drug candidates.

### IV. EXAMPLE

The following Example has been included to provide guidance to one of ordinary skill in the art for practicing representative embodiments of the presently disclosed subject matter. In light of the present disclosure and the general level of skill in the art, those of skill can appreciate that the following Example is intended to be exemplary only and that numerous changes, modifications, and alterations can be employed without departing from the scope of the presently disclosed subject matter.

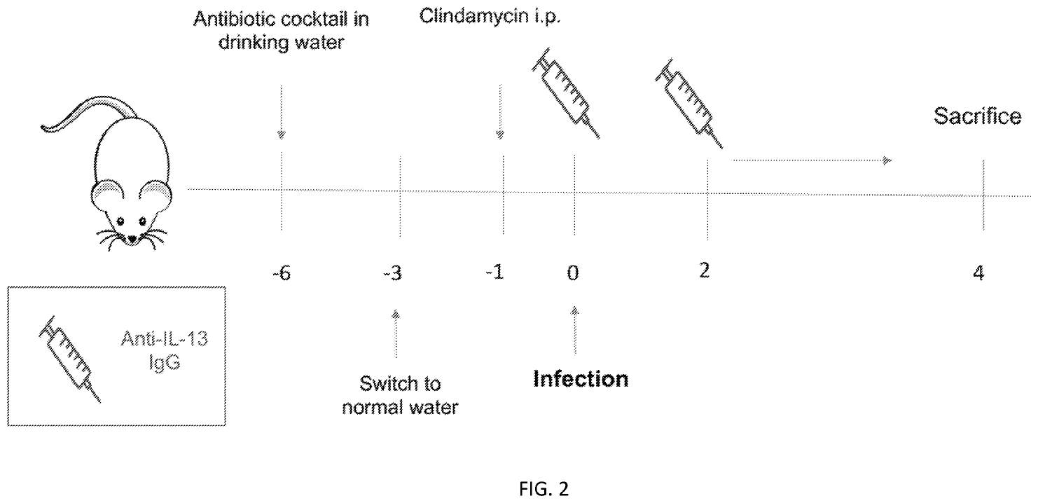

Methods: Mice were given three days of antibiotics in their drinking water, followed by two days of rest. One day before infection, mice were given i.p. injection of clindamycin. Mice were then infected with spores of the R20291 strain of C. difficile. For anti-IL-13 experiments, mice were given i.p. injections of anti-IL-13 (catalog # 16-7135-81, Thermo Fisher Scientific, Waltham, Mass., United States of America) or IgG antibodies on days 0 and 2. See FIG. 2. For flow cytometry, colons were separated into single cells, stained with fluorescent antibodies, then analyzed via fluorescence-activated cell sorting (FACS).

In a further study, groups of mice were given three days of antibiotics in their drinking water, followed by two days of rest. One day before infection, mice were given i.p. injection of clindamycin. Mice were then infected with spores of the R20291 strain of C. difficile. For exogenous IL experiments, mice were given i.p. injections of recombinant IL-13 (5 μg, catalog # 210-13, PeproTech, Inc., Rocky Hill, N.J., United States of America), recombinant IL-4 (catalog # 214-14, PeproTech, Inc., Rocky Hill, N.J., United States of America) or PBS on days 0 and 1. See FIG. 5.

In addition, mice were treated according to one of the following protocols: neither antibiotics or C. difficile (control group), antibiotics only (antibiotics group), or antibiotics and infected with 500 spores of R20291. Mice were sacrificed on different days post infection (day 2, day 4 or day 5), and cecal levels of IL-13Ra2, IL-13, and IL-10 were measured by ELISA.

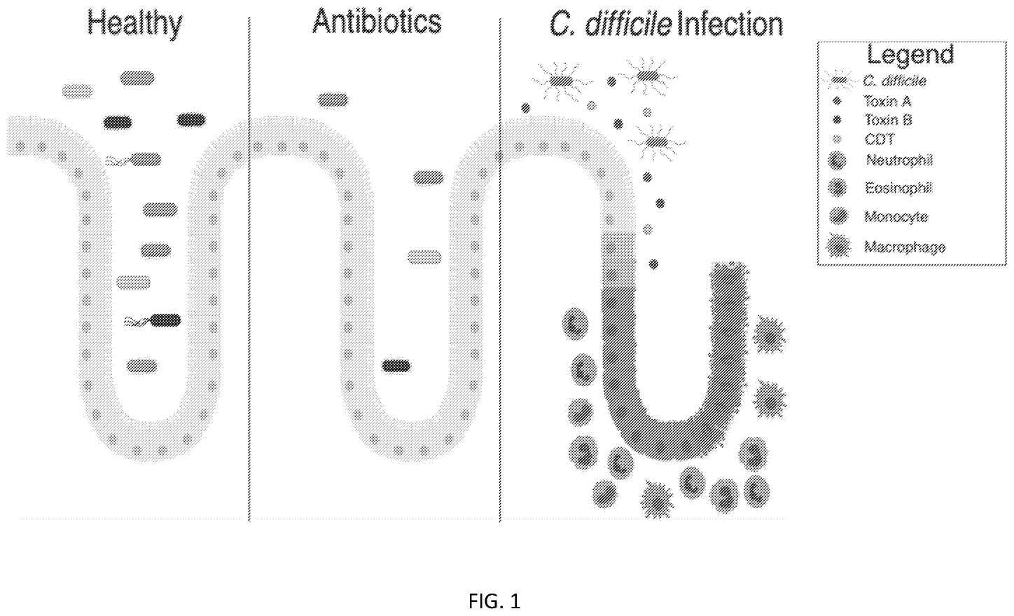

Results: Antibiotics disrupt the normal microbiota, allowing a niche in which C. difficile can germinate and reproduce. See FIG. 1. Infection results in high amounts of type 17 inflammation, involving neutrophils, monocytes, and cytokines. In excess, this can lead to host damage. Protective immune responses include type 2 responses (allergy/asthma responses).^{1,2}

Protective IL-33 treatment leads to increases in IL-4 and IL-13, produced by ILC2s (and eosinophils). ILC2s are necessary for IL-33 mediated protection. IL-4 and IL-13 are important for downstream adaptive type 2 response, epithelial remodeling, and macrophage polarization.³ Anti-IL-4 treatment results in delayed recovery.¹

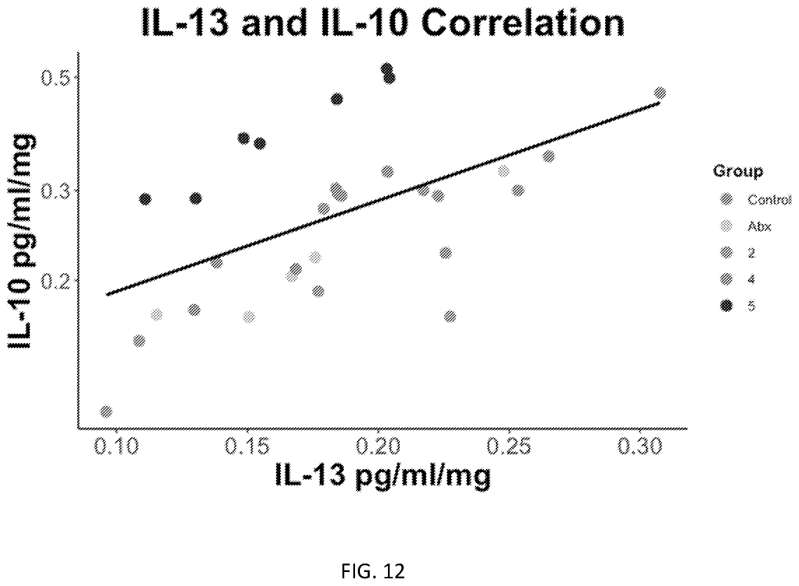

As shown in FIG. 12, IL-13 expression during recovery from CDI strongly correlates with that of the anti-inflammatory cytokine IL-10. IL-10 has been shown previously to protect from CDI and is thought to be primarily produced by macrophages during innate immune responses. IL-13 is also known to promote the production of IL-10 by macrophages. Here, IL-13 correlated strongly with IL-10, which, without being bound to any one theory, suggests that they can be involved in the same pathway.

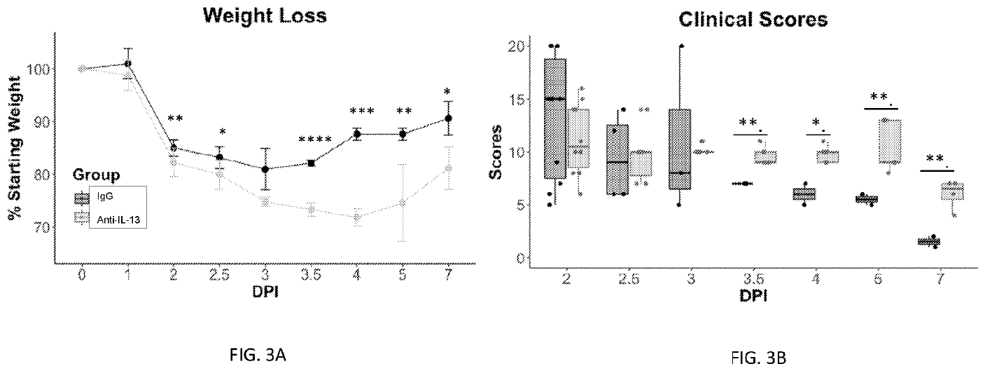

Mice infected with 10⁴ R20291 spores and administered either neutralizing IL-13 (anti-IL-13) or IgG control on days 0 and 2 were monitored for weight loss (see FIG. 3A) and clinical score. See FIG. 3B. Mice who received neutralizing IL-13 antibodies had increased weight loss and delayed recovery from disease. IL-13 can act directly upon the epithelium, while IL-4 cannot, suggesting that anti-IL-13 weight loss can be due to increased epithelial damage. IL-4 and IL-13 have similar roles on immune cells, suggesting that overlapping phenotype of delayed recovery can be immune-mediated.

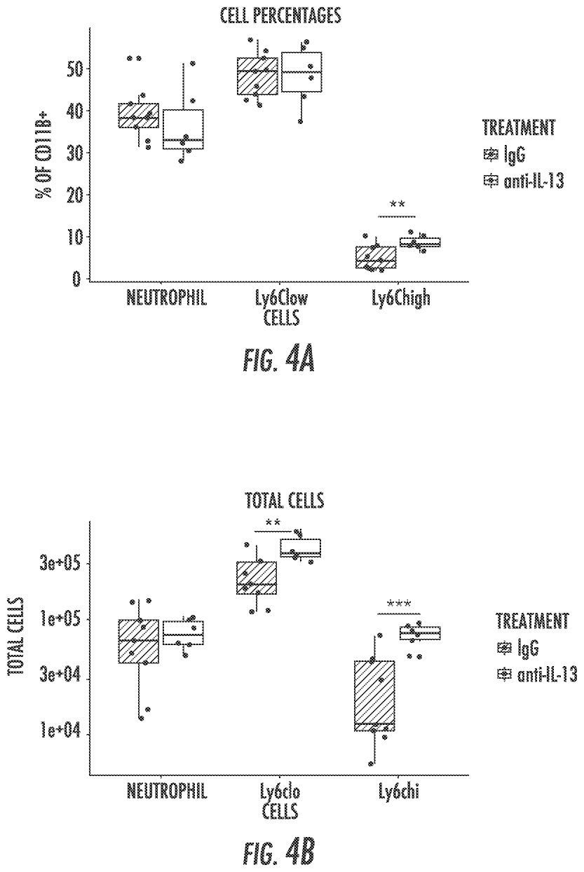

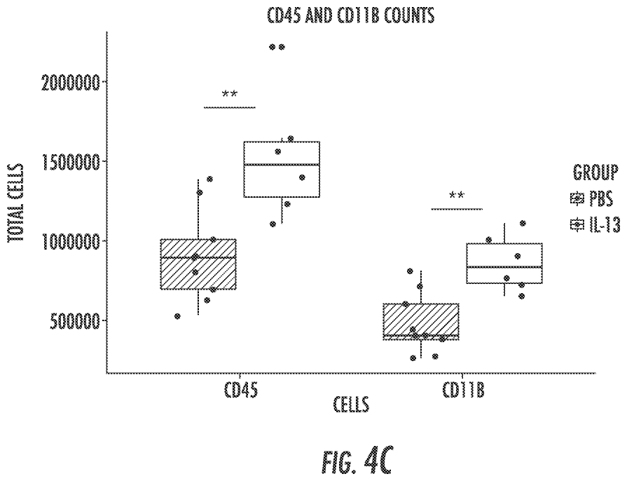

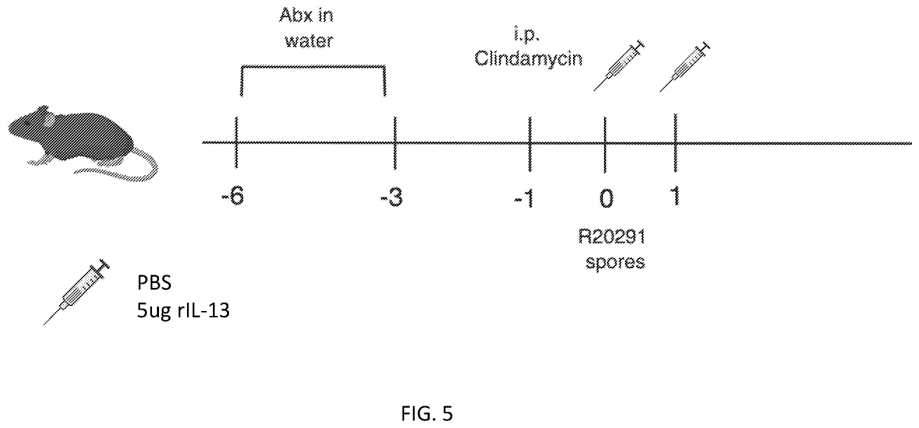

Mice infected with 500 spores of R20291 and administered either anti-IL-13 or IgG control i.p. injections on days 0 and 2 were sacrificed on day 4 following infection, and their colon cells were stained and analyzed via FACS. Neutrophils, Ly6C-low and -high monocytes percent of total CD11B+ myeloid cells are shown in FIG. 4A and total numbers are shown in FIG. 4B. Total cell numbers of CD45+ and CD11B+ cells are shown in FIG. 4C.

Anti-IL-13 resulted in increased Ly6C^{hi} monocyte proportions and increased monocyte total cells. Ly6C monocytes can engraft into tissue-resident macrophages. Without being bound to any one theory, it is believed that IL-13 can promote this transition, potentially into alternatively-activated macrophage (AAM) phenotypes. Anti-IL-13 results in high cell numbers of both total CD45 and myeloid CD11B+ cells on day 4 of infection. This can be the result of increased recruitment or impaired inflammation clearance.

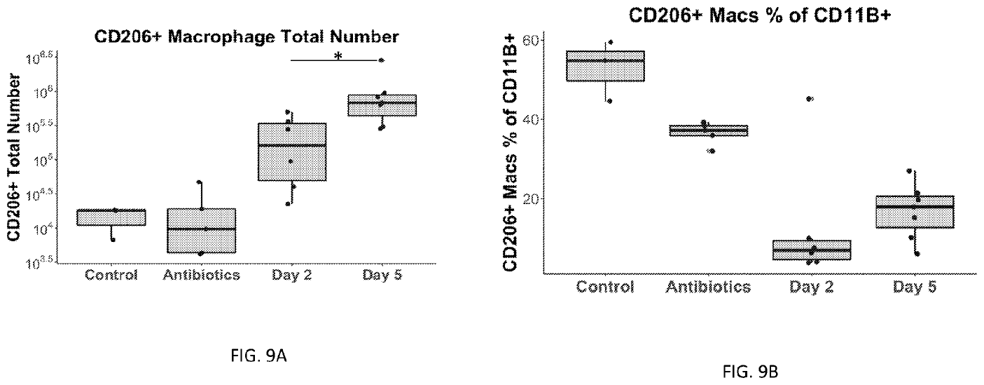

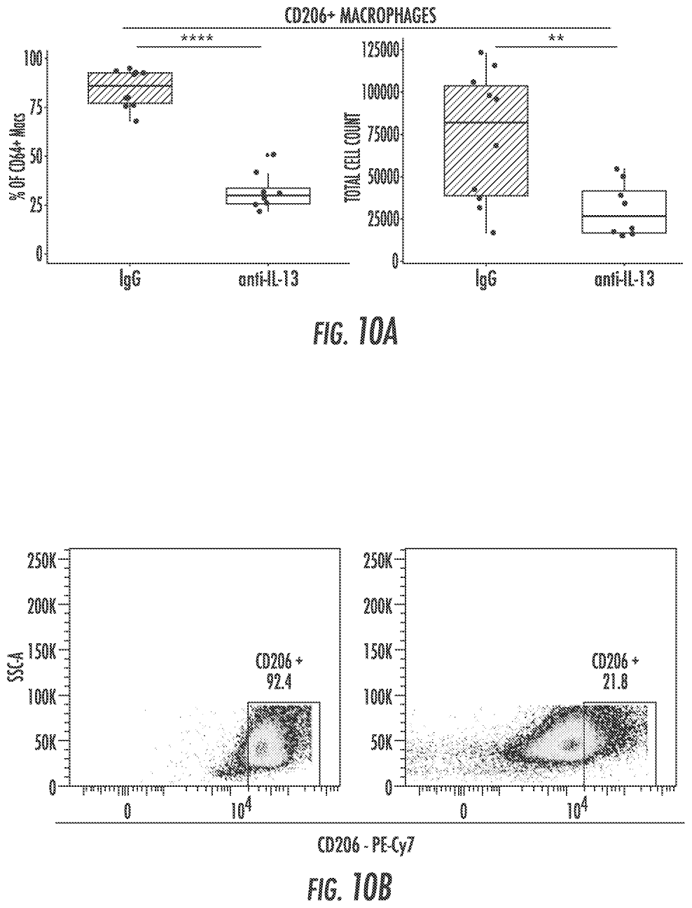

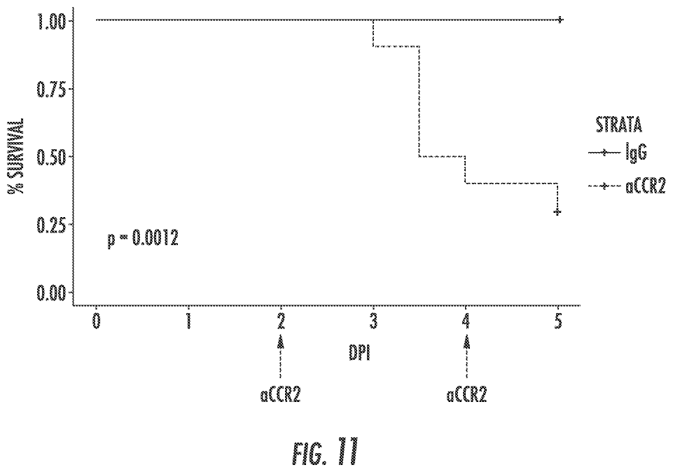

Anti-IL-13 also reduced CD206+ macrophages, which are associated with disease recovery. CD206+ macrophages are generally considered anti-inflammatory and pro-tissue repair and can be induced by IL-13. During CDI, they are increased during the recovery period (day 5) (see FIGS. 9A and 9B), which, without being bound to any one theory suggest that this time period could be when these cells are promoting host recovery from inflammation and tissue damage. Anti-IL-13, which is associated with worse outcomes in disease, reduces these cells by 3 days post-infection. See FIGS. 10A and 10B. Accordingly, and again without being bound to any one theory, this reduction can be one mechanism through how IL-13 neutralization prevents recovery.

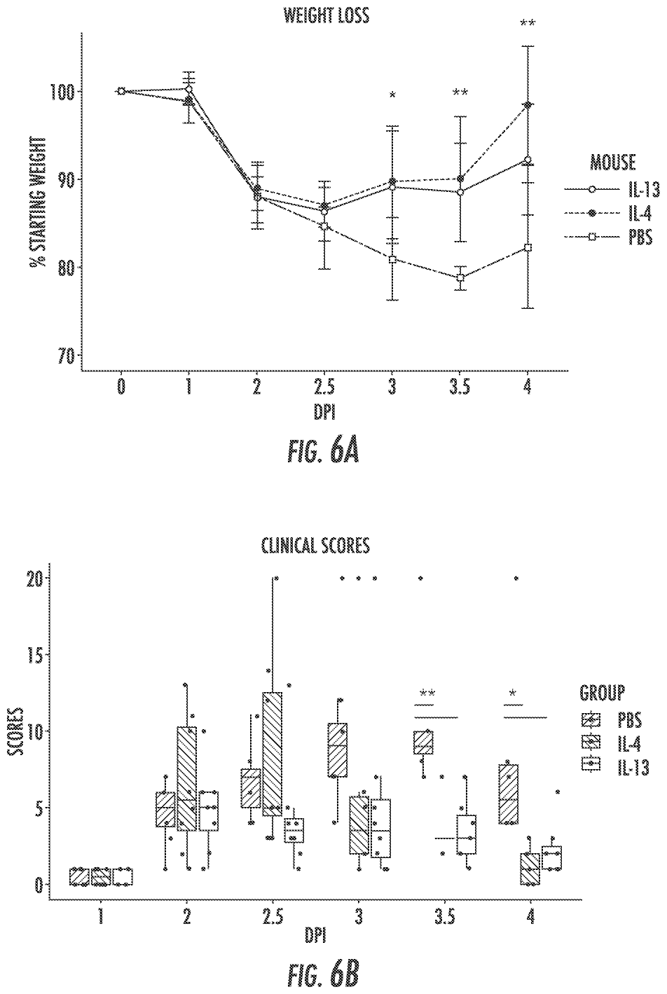

Mice infected with 10⁴ R20291 spores and administered either IL-13, IL-4 or PBS control on days 0 and 1 were monitored for weight loss (see FIG. 6A) and clinical score. See FIG. 6B. Mice who received IL-13 and IL-4 had decreased weight loss and faster recovery from disease. IL-13 can also promote CD206+ macrophages polarization from tissue-recruited CCR2+ monocytes. When these monocytes are depleted starting on day 2 post-infection, mice have significantly worse disease. See FIG. 11. This result could support that tissue-recruited monocytes are giving rise to anti-inflammatory macrophages in an IL-13 dependent manner.

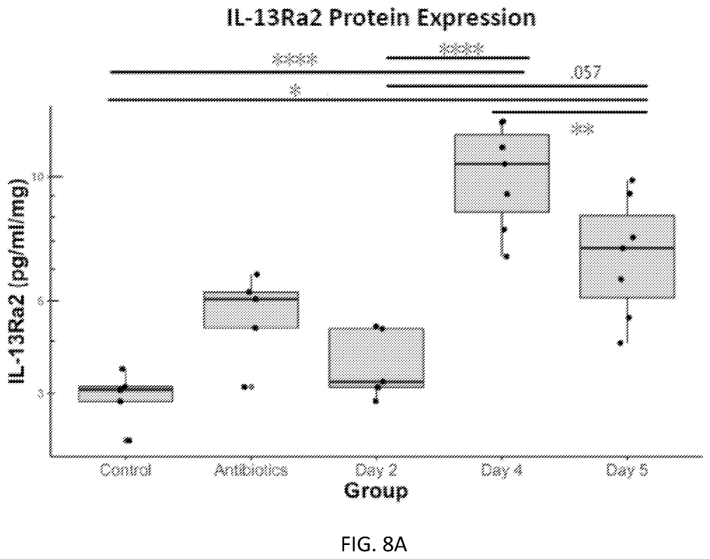

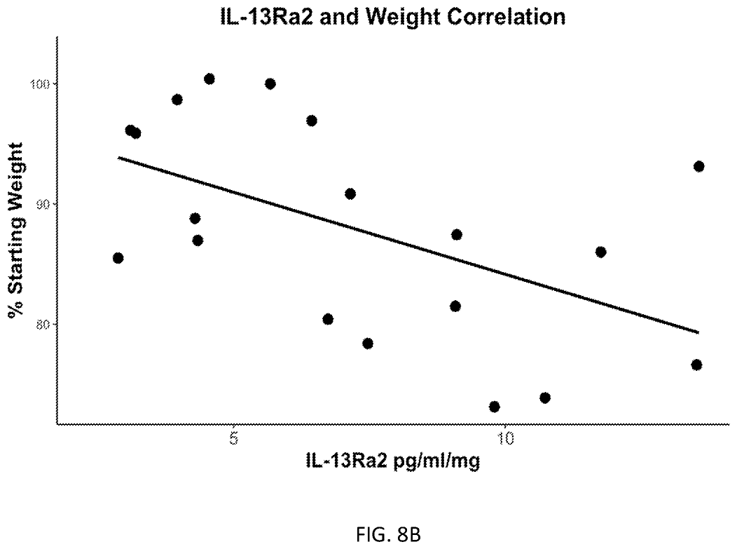

The IL-13ra2 protein levels in mice treated with antibiotics and infected with 500 spores of R20291 were measured on days 2, 4, and 5 after infection. See FIG. 8A. Without being bound to any one theory, the results could suggest at which time point IL-13 signaling is highest, as IL-13Ra2 should track with IL-13 levels. IL-13Ra2 protein levels were also compared to weight and found to correlate negatively with weight. See FIG. 8B.

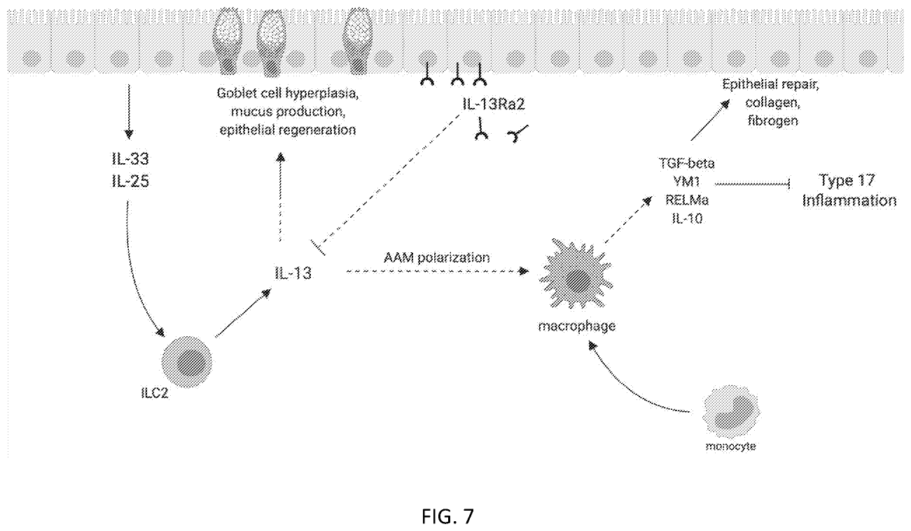

Discussion: Anti-IL-4 and anti-IL-13 treatment leads to delayed recovery from CDI. Without being bound to any one theory, this can be through similar mechanisms because of overlapping roles of these cytokines. Anti-IL-13 leads to increased weight loss, likely through the direct role of IL-13 on the epithelium. The results suggest that IL-13 can promote the transition of Ly6C ^{hi} monocytes into resident macrophages and that IL-13 can decrease recruitment or increase clearance of immune cells to site of infection by day 4. See FIG. 7.

Based on these results, IL-4 and/or IL-13 administration can be protective and/or promote recovery. In addition, use of neutralizing antibodies against IL-13's decoy receptor, IL-13Ra2, can serve as a treatment for promoting IL-13's beneficial effects. Ly6C^{hi} monocytes can transition into M2-like macrophages. Additional studies are performed to determine if these cells promote recovery. Other studies are performed to determine what is downstream of these cytokines (e.g., TGF-β, YM1, IL-10, etc.) and to observe whether timing of these cytokines (early or late during infection) influences their roles. Finally, other studies are performed to determine if the role of these cytokines is directly downstream of eosinophils and ILC2s, which produce them.

### REFERENCES

The references listed below as well as all references cited in the specification are incorporated herein by reference to the extent that they supplement, explain, provide a background for or teach methodology, techniques and/or compositions employed herein. All cited patents and publications referred to in this application are herein expressly incorporated by reference.
1. Buonomo et al. (2016) “Mitobiota-regulated IL-25 Increases Eosinophil Number to Provide Protection During Clostridium difficile Infection,” Cell Reports, 16(2), 432-443.
2. Cowardin et al. (2016) “The Binary Toxin CDT Enhances Clostridium difficile Virulence by Suppressing Protective Colonic Eosinophilia,” Nature Microbiology, 1(8), 16108.
3. Wills-Karp and Finkelman (2008) “Untangling the Complex Web IL-4- and IL-13-mediated Signaling Pathways,” Science Signaling, 1(51), pe55-pe55.

It will be understood that various details of the presently disclosed subject matter may be changed without departing from the scope of the presently disclosed subject matter. Furthermore, the foregoing description is for the purpose of illustration only, and not for the purpose of limitation.

1. A method of treating or preventing a Clostridium difficile (C. difficile) infection, or a symptom or pathology associated therewith, in a subject in need thereof, the method comprising administering to the subject a composition that comprises a therapeutically effective amount of at least one agent that enhances a biological activity of interleukin-13 (IL-13) in the subject.

2. The method of claim 1, wherein the at least one agent that enhances a biological activity of IL-13 comprises an IL-13 peptide or a fragment or homolog thereof.

3. The method of claim 2, wherein the IL-13 peptide is a peptide having an amino acid sequence that is at least 95% identical to SEQ ID NO: 1, SEQ ID NO: 2, or SEQ ID NO: 3.

4. The method of claim 1, wherein the at least one agent that enhances an activity of IL-13 comprises an agent that blocks interleukin-13 receptor subunit alpha-2 (IL-13Ra2).

5. The method of claim 4, wherein the agent that blocks IL-13Ra2 is an antibody that binds to IL-13Ra2, optionally wherein the antibody that binds to IL-13Ra2 is a monoclonal antibody.

6. The method of claim 1, wherein the administering results in a more rapid recovery from a C. difficile infection as compared to the recovery expected in the absence of administering the composition to the subject.

7. The method of claim 1, wherein the administering prevents a recurrence of a C. difficile infection.

8. The method of claim 1, wherein the subject is a mammal.

9. The method of claim 1, wherein the subject is a human.

10. The method of claim 1, wherein the subject has a C. difficile infection.

11. The method of claim 1, wherein the subject has one or more increased risk factors for a C. difficile infection, optionally wherein the one or more increased risk factors are selected from the group consisting of hospitalization, antibiotic use, and increased age.

12. The method of claim 1, wherein administering the composition inhibits weight loss.

13. The method of claim 1, wherein administering the composition inhibits diarrhea.

14. The method of claim 1, wherein administering the composition inhibits colonic inflammation.

15. The method of claim 1, further comprising administering at least one additional therapeutic agent to the subject, optionally wherein the composition comprises the at least one additional therapeutic agent.

16. The method of claim 15, wherein said at least one additional therapeutic agent is selected from the group consisting of an anesthetic, an analgesic, an antimicrobial agent, a steroid, a growth factor, a cytokine other than IL-13, and an anti-inflammatory agent.

17. The method of claim 16, wherein said at least one additional therapeutic agent comprises a cytokine other than IL-13, optionally an interleukin other than IL-13, further optionally wherein said at least one additional therapeutic agent comprises interleukin-4 (IL-4), interleukin-33 (IL-33), or interleukin-25 (IL-25).

18. The method of claim 16, wherein said at least one additional therapeutic agent comprises an antimicrobial agent, optionally wherein said antimicrobial agent is selected from the group consisting of an antibacterial agent, an antifungal agent, and an antiviral agent.

19. The method of claim 18, wherein said antimicrobial agent comprises at least one antibiotic selected from the group consisting of vancomycin, fidaxomicin, metronidazole, nitazoxanide, and rifaximin.

20. A composition for use in treating or preventing a Clostridium difficile (C. difficile) infection, or a symptom or pathology associated therewith, wherein the composition comprises a therapeutically effective amount of at least one agent that enhances a biological activity of interleukin-13 (IL-13).

21. The composition of claim 20, wherein the agent that enhances a biological activity of IL-13 comprises an IL-13 peptide or a fragment or homolog thereof, optionally wherein the IL-13 peptide comprises an amino acid sequence that is at least 95% identical to SEQ ID NO: 1, SEQ ID NO: 2, or SEQ ID NO: 3.

22. The composition of claim 20, wherein the agent that enhances a biological activity of IL-13 comprises an agent that blocks interleukin-13 receptor subunit alpha-2 (IL-13Ra2), optionally wherein said agent is an anti-IL-13Ra2 antibody.

23. A method of treating or preventing a Clostridium difficile (C. difficile) infection, or a symptom or pathology associated therewith, in a subject in need thereof, the method comprising administering to the subject a composition that comprises a therapeutically effective amount of an interleukin-4 (IL-4) peptide, or a fragment or a homolog thereof, optionally wherein the IL-4 peptide comprises an amino acid sequence that is at least 95% identical to SEQ ID NO: 4, SEQ ID NO: 5, SEQ ID NO: 6, or SEQ ID NO: 7.

24. A composition for use in treating or preventing a Clostridium difficile (C. difficile) infection, or a symptom or pathology associated therewith, wherein the composition comprises a therapeutically effective amount of an interleukin-4 (IL-4) peptide, or a fragment or a homolog thereof, optionally wherein the IL-4 peptide comprises an amino acid sequence that is at least 95% identical to SEQ ID NO: 4, SEQ ID NO: 5, SEQ ID NO: 6, or SEQ ID NO: 7.
